(19) United States
(12) Patent Application Publication
(10) Pub. No.: US 2025/0170077 A1
(43) Pub. Date: May 29, 2025

(54) Treatment of cutaneous neurofibromas with Mirdametinib

(71) Applicant: SpringWorks Therapeutics, Inc., Stamford, CT (US)

(72) Inventors: Uchenna H. Iloeje, Stamford, CT (US); Abraham J Langseth, Stamford, CT (US)

(21) Appl. No.: 18/841,882

(22) PCT Filed: Mar. 16, 2023
(86) PCT No.: PCT/US2023/064572

§ 371 Date: Aug. 27, 2024

Related U.S. Application Data
(60) Provisional application No. 63/321,036, filed on Mar. 17, 2022.
(60) Provisional application No. 63/321,046, filed on Mar. 17, 2022.

(51) Int. Cl. A61K 31/166; A61P 35/00

(52) U.S. Cl. CPC A61K 31/166; A61P 35/00

(57) ABSTRACT

The present disclosure relates to methods for treating cutaneous neurofibromas (cNF) comprising administering to a patient in need thereof mirdametinib or a pharmaceutically acceptable salt thereof.

This application is the U.S. national stage of International Patent Application No. PCT/US2023/064572, filed Mar. 16, 2023, which claims the benefit of U.S. Provisional Application No. 63/321,036, filed Mar. 17, 2022, and U.S. Provisional Application No. 63/321,046, filed Mar. 17, 2022, each of which is hereby incorporated by reference.

### FIELD OF THE INVENTION

Mirdametinib is an allosteric, small molecule targeting mitogen-activated protein kinase kinase (MEK).

Weiss describes a Phase II clinical trial of mirdametinib in subjects with neurofibromatosis type 1 who have a plexiform neurofibroma (Weiss et al., J. Clin. Oncol., 29, 797-806, 2021).

The present disclosure relates to methods for treating cutaneous neurofibromas (cNF) comprising administering to a patient in need thereof mirdametinib or a pharmaceutically acceptable salt thereof.

### BACKGROUND

There are many clinical features associated with the neurogenetic condition neurofibromatosis type 1 (NF1). However, the feature that affects the majority of NF1 patients is the development of cutaneous neurofibromas (cNF). Neurofibromas are defined as histologically benign (WHO grade I) tumors composed of multiple cell types including Schwann cells, fibroblasts, immune cells (such as mast cells and macrophages), and other elements of nerve. Regardless of their location, all neurofibromas share certain histologic and cellular characteristics.

The most common and prominent location of neurofibromas is the skin (including the epidermis and dermis). Discrete lesions are often referred to as dermal or cutaneous neurofibromas. cNF are benign and are not known to have any malignant potential.

Although not life-threatening, cNF have a major effect upon quality of life for most patients with NF1 due to their prevalence and disfigurement. Furthermore, the number of cNF increases with age after adolescence and throughout a patient's lifespan. Despite the high prevalence of cNF in people with NF1 and their documented influence on quality of life, current treatment is limited to surgical removal or physical destruction.

There is a need for improved therapies for cutaneous neurofibromas.

### BRIEF SUMMARY OF THE INVENTION

One aspect of the present invention is a method for treating cutaneous neurofibromas (cNF) in a human patient comprising administering to the patient mirdametinib or a pharmaceutically acceptable salt thereof. In some aspects, the patient in need thereof additionally has neurofibromatosis 1 (“NF1”)

In some aspects, a therapeutically effective amount of mirdametinib, or a pharmaceutically acceptable salt thereof, is administered. In some aspects, the mirdametinib, or a pharmaceutically acceptable salt thereof, is administered in an amount of about 1 mg/m² to about 10 mg/m² per day based on mirdametinib free base. In some aspects, the mirdametinib, or a pharmaceutically acceptable salt thereof, is administered in an amount of about 1 mg to about 10 mg per day based on mirdametinib free base.

In some aspects, the mirdametinib, or a pharmaceutically acceptable salt thereof, is administered in a single dosage form comprising about 0.1 mg/m² to about 10 mg/m² based on mirdametinib free base. In some aspects, the mirdametinib, or a pharmaceutically acceptable salt thereof, is administered in a single dosage form comprising about 0.1 mg to about 10 mg based on mirdametinib free base.

In some aspects, the mirdametinib, or a pharmaceutically acceptable salt thereof, is administered once daily. In some aspects, the mirdametinib, or a pharmaceutically acceptable salt thereof, is administered twice daily.

In one embodiment, the mirdametinib, or a pharmaceutically acceptable salt thereof, is orally administered in an amount of about 1 mg/m² to about 10 mg/m² per day based on mirdametinib free base.

In another embodiment, the mirdametinib, or a pharmaceutically acceptable salt thereof, is orally administered in an amount of about 1 mg to about 10 mg per day based on mirdametinib free base.

In yet another embodiment, the mirdametinib, or a pharmaceutically acceptable salt thereof, is orally administered in an amount of about 2 mg/m² per day based on mirdametinib free base.

In yet another embodiment, the mirdametinib, or a pharmaceutically acceptable salt thereof, is orally administered in an amount of about 2 mg per day based on mirdametinib free base.

In yet another embodiment, the mirdametinib, or a pharmaceutically acceptable salt thereof, is orally administered in an amount of about 4 mg per day based on mirdametinib free base.

In yet another embodiment, the mirdametinib, or a pharmaceutically acceptable salt thereof, is orally administered in an amount of about 6 mg per day based on mirdametinib free base.

In yet another embodiment, the mirdametinib, or a pharmaceutically acceptable salt thereof, is orally administered in an amount of about 8 mg per day based on mirdametinib free base.

In yet another embodiment, the mirdametinib, or a pharmaceutically acceptable salt thereof, is orally administered twice daily in an amount of about 1 mg based on mirdametinib free base.

In yet another embodiment, the mirdametinib, or a pharmaceutically acceptable salt thereof, is orally administered twice daily in an amount of about 2 mg based on mirdametinib free base.

In yet another embodiment, the mirdametinib, or a pharmaceutically acceptable salt thereof, is orally administered twice daily in an amount of about 3 mg based on mirdametinib free base.

In yet another embodiment, the mirdametinib, or a pharmaceutically acceptable salt thereof, is orally administered twice daily in an amount of about 4 mg based on mirdametinib free base.

In one embodiment of any of the methods described herein,
(a) for a patient having a body surface area no more than 0.69 m², the patient is initially orally administered 1 mg mirdametinib twice daily,
(b) for a patient having a body surface area of 0.7 to 1.04 m², the patient is initially orally administered 2 mg mirdametinib twice daily,
(c) for a patient having a body surface area of 1.05 to 1.49 m², the patient is initially orally administered 3 mg mirdametinib twice daily, and
(d) for a patient having a body surface area of at least 1.5 m², the patient is initially orally administered 4 mg mirdametinib twice daily.

In one embodiment, the maximum daily dose is 4 mg mirdametinib twice daily.

In one embodiment, over each four week period, the mirdametinib is administered for the first three weeks and discontinued for the last one week.

In another embodiment, the mirdametinib is administered daily without interruption.

In one embodiment of any of the methods described herein, the dose administered is reduced due to an adverse event, wherein the dose is reduced as follows:
(a) if the dose at the time of the event is 1 mg mirdametinib twice daily, then the reduced daily dose is 1 mg orally administered in the morning only;
(b) if the dose at the time of the event is 2 mg mirdametinib twice daily, then the reduced daily dose is 2 mg orally administered in the morning and 1 mg administered in the afternoon or evening;
(c) if the dose at the time of the event is 3 mg mirdametinib twice daily, then the reduced daily dose is 2 mg orally administered twice daily; and
(d) if the dose at the time of the event is 4 mg mirdametinib twice daily, then the reduced daily dose is 3 mg orally administered twice daily. In one embodiment, the adverse event resulting in the dose reduction is acneiform.

In one embodiment of any of the methods described herein, the method further comprises prior to treatment (i) determining whether to select mirdametinib as a treatment for the patient, and (ii) selecting mirdametinib as a treatment for the patient at least partially based on its objective response rate, where the objective response rate is defined as at least a 20% decrease in tumor size using centrally read MRI volumetric analysis. In one embodiment, in step (i), mirdametinib is selected based on a response rate of at least 70%. In another embodiment, in step (i), mirdametinib is selected based on a response rate of at least 75%. In yet another embodiment, in step (i), mirdametinib is selected based on a response rate of at least 80%. In yet another embodiment, in step (i), mirdametinib is selected based on a response rate of at least 85%. In yet another embodiment, in step (i), mirdametinib is selected based on a response rate of at least 90%. In yet another embodiment, in step (i), mirdametinib is selected based on a response rate of at least 95%.

In some aspects, the mirdametinib, or a pharmaceutically acceptable salt thereof, exhibits high blood-brain-barrier penetration.

In one embodiment, the patient has the clinical diagnosis of NF1 using the NIH Consensus Conference and one or more of the following:
(a) six or more café-au-lait macules with a diameter >5 mm in prepubertal and >15 mm in post-pubertal individuals;
(b) freckling in axilla or inguinal regions;
(c) optic glioma;
(d) two or more Lisch nodules;
(e) a distinctive bony lesion (dysplasia of the sphenoid bone or dysplasia of thinning of long bone cortex); and
(f) a first degree relative with NF1.

In another embodiment, the patient has a constitutional NF1 mutation documented in a Clinical Laboratory Improvement Amendments/College of American Pathologists certified lab.

In one embodiment of any of the methods described herein, the patient is a human. In one embodiment, the human is an adult. In another embodiment, the patient is 2 to 15 years of age.

In one embodiment of any of the methods described herein, the human has had no prior exposure to MEK inhibitors.

In one embodiment of any of the methods described herein, the mirdametinib, or a pharmaceutically acceptable salt thereof, is administered orally. In some aspects, the mirdametinib, or a pharmaceutically acceptable salt thereof, is dispersible in a potable liquid or orodispersible in a patient's saliva. In some aspects, the mirdametinib, or a pharmaceutically acceptable salt thereof, is administered orally as a solid dosage form. In some aspects, the solid dosage form is a tablet or capsule. In some aspects, the solid dosage form is a capsule.

In one embodiment of any of the methods described herein, the mirdametinib, or a pharmaceutically acceptable salt thereof, is administered as a monotherapy to cNF. In one embodiment of any of the methods described herein, the mirdametinib, or a pharmaceutically acceptable salt thereof, is administered in combination with another active ingredient and/or surgery to treat cNF.

### DETAILED DESCRIPTION OF THE INVENTION

### I. Definitions

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “mirdametinib” refers to the single enantiomer N-((R)-2,3-dihydroxypropoxy)-3,4-difluoro-2-(2-fluoro-4-iodo-phenylamino) benzamide (free base form). The teachings throughout the specification regarding mirdametinib equally apply to pharmaceutically acceptable salts of mirdametinib.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject. The patient may be a pediatric patient.

The term “mg/m²” refers to the dose in milligrams per m2 body surface area of the patient.

The term “pediatric” refers to a human subject under the age of 21 years at the time of treatment. The term “pediatric” can be further divided into various subpopulations including: neonates (from birth through the first 28 days of life); infants (29 days of age to less than two years of age); children (two years of age to less than 12 years of age); and adolescents (12 years of age through 21 years of age (up to, but not including, the twenty-second birthday)). See, e.g., Berhman R E, Kliegman R, Arvin A M, Nelson W E. Nelson Textbook of Pediatrics, 15th Ed. Philadelphia: W. B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. Younger pediatric patients in particular, such as neonates, infants and young children, can have difficulty swallowing whole capsules or tablets.

The term “dispersible” as used herein refers to a composition (e.g., a tablet, powder, granules, minitablets, or pellets) which disintegrates and/or dissolves when combined with water or another potable liquid (e.g., a non-water beverage), or a subject's own saliva when placed in the subject's mouth, with or without the addition of agitation or temperature modification. In some aspects, the dispersible composition disintegrates or dissolves within 10 minutes, 9 minutes, 8 minutes, 7 minutes, 6 minutes, 5 minutes, 4 minutes, 3 minutes, 2 minutes, or 1 minute after being combined with water or another potable liquid. Such disintegration or dissolution need not be complete. For example, a dispersible tablet may dissolve almost entirely, but some undissolved particulate matter may remain. Dispersible formulations of mirdametinib suitable for the methods described herein include those described in U.S. Pat. No. 11,571,402, which is hereby incorporated by reference.

The term “orodispersible” refers to a composition which is capable of dissolving or disintegrating in a subject's mouth (i.e., dissolving or disintegrating in a subject's saliva) if administered orally, without a requirement of first dissolving or disintegrating in a separate container.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Thus, those in need of treatment include those already diagnosed with or suspected of having the disorder. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. The term “therapeutically effective amount” is meant to include the amount of a compound that, when administered, is sufficient to prevent development of, or alleviate to some extent, one or more of the symptoms of a disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

In certain aspects, a subject is successfully “treated” for a tumor, according to the methods described herein if the patient shows one or more of the following: a reduction in the size of the tumor; relief of one or more symptoms associated with the specific tumor; a reduction in the volume of the tumor; improvement in quality of life; increased progression-free survival (PFS), disease-free survival (DFS), overall survival (OS), metastasis-free survival (MFS), complete response (CR), minimal residual disease (MRD), partial response (PR), stable disease (SD), a decrease in progressive disease (PD), an increased time to progression (TTP), or any combination thereof. In some aspects, nationally or internationally accepted standards of treatment outcomes in a given tumor can be used to determine whether an effective amount of mirdametinib meets any of these particular endpoints (e.g., CR, PFS, PR).

The terms “pharmaceutically acceptable carrier,” “pharmaceutically acceptable excipient,” “physiologically acceptable carrier,” or “physiologically acceptable excipient” refer to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, FL, 2004 (incorporated herein by reference).

The term “pharmaceutically acceptable salts” refers to the relatively non-toxic, inorganic and organic acid addition salts of Compound A or Compound B. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts. See, e.g., Berge et al. (1977) “Pharmaceutical Salts”, J. Pharm. Sci. 66:1-19.

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, and nitric acid; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, and isothionic acid.

The terms “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

### II. Methods of Treatment

Methods for treating a patient having cutaneous neurofibromas (“cNF”), comprising administering to a patient in need thereof mirdametinib or a pharmaceutically acceptable salt thereof are provided herein. In some aspects, the patient in need thereof additionally has neurofibromatosis 1 (“NF1”).

In some aspects, a therapeutically effective amount of mirdametinib, or a pharmaceutically acceptable salt thereof, is administered.

In some aspects, the mirdametinib, or a pharmaceutically acceptable salt thereof, is administered in an amount of about 1 mg/m² to about 10 mg/m² per day based on mirdametinib free base, about 1.5 mg/m² to about 9.5 mg/m² per day based on mirdametinib free base, about 2 mg/m² to about 9 mg/m² per day based on mirdametinib free base, about 2.5 mg/m² to about 8.5 mg/m² per day based on mirdametinib free base, about 3 mg/m² to about 8 mg/m² per day based on mirdametinib free base, about 3.5 mg/m² to about 7.5 mg/m² per day based on mirdametinib free base, about 4 mg/m² to about 7 mg/m² per day based on mirdametinib free base, about 4.5 mg/m² to about 6.5 mg/m² per day based on mirdametinib free base, or about 5 mg/m² to about 6 mg/m² per day based on mirdametinib free base. In some aspects, the mirdametinib, or a pharmaceutically acceptable salt thereof, is administered in an amount of about 1 mg/m² per day based on mirdametinib free base, about 1.5 mg/m² per day based on mirdametinib free base, about 2 mg/m² per day based on mirdametinib free base, about 2.5 mg/m² per day based on mirdametinib free base, about 3 mg/m² per day based on mirdametinib free base, about 3.5 mg/m² per day based on mirdametinib free base, about 4 mg/m² per day based on mirdametinib free base, about 4.5 mg/m² per day based on mirdametinib free base, about 5 mg/m² per day based on mirdametinib free base, about 5.5 mg/m² per day based on mirdametinib free base, about 6 mg/m² per day based on mirdametinib free base, about 6.5 mg/m² per day based on mirdametinib free base, about 7 mg/m² per day based on mirdametinib free base, about 7.5 mg/m² per day based on mirdametinib free base, about 8 mg/m² per day based on mirdametinib free base, about 8.5 mg/m² per day based on mirdametinib free base, about 9 mg/m² per day based on mirdametinib free base, about 9.5 mg/m² per day based on mirdametinib free base, or about 10 mg/m² per day based on mirdametinib free base.

In some aspects, the mirdametinib, or a pharmaceutically acceptable salt thereof, is administered in an amount of about 1 mg to about 10 mg per day based on mirdametinib free base, about 1.5 mg to about 9.5 mg per day based on mirdametinib free base, about 2 mg to about 9 mg per day based on mirdametinib free base, about 2.5 mg to about 8.5 mg per day based on mirdametinib free base, about 3 mg to about 8 mg per day based on mirdametinib free base, about 3.5 mg to about 7.5 mg per day based on mirdametinib free base, about 4 mg to about 7 mg per day based on mirdametinib free base, about 4.5 mg to about 6.5 mg per day based on mirdametinib free base, or about 5 mg to about 6 mg per day based on mirdametinib free base. In some aspects, the mirdametinib, or a pharmaceutically acceptable salt thereof, is administered in an amount of about 1 mg per day based on mirdametinib free base, about 1.5 mg per day based on mirdametinib free base, about 2 mg per day based on mirdametinib free base, about 2.5 mg per day based on mirdametinib free base, about 3 mg per day based on mirdametinib free base, about 3.5 mg per day based on mirdametinib free base, about 4 mg per day based on mirdametinib free base, about 4.5 mg per day based on mirdametinib free base, about 5 mg per day based on mirdametinib free base, about 5.5 mg per day based on mirdametinib free base, about 6 mg per day based on mirdametinib free base, about 6.5 mg per day based on mirdametinib free base, about 7 mg per day based on mirdametinib free base, about 7.5 mg per day based on mirdametinib free base, about 8 mg per day based on mirdametinib free base, about 8.5 mg per day based on mirdametinib free base, about 9 mg per day based on mirdametinib free base, about 9.5 mg per day based on mirdametinib free base, or about 10 mg per day based on mirdametinib free base.

In some aspects, the mirdametinib, or a pharmaceutically acceptable salt thereof, is administered in a single dosage form comprising about 0.1 mg/m² to about 10 mg/m² based on mirdametinib free base, about 0.5 mg/m² to about 9.5 mg/m² based on mirdametinib free base, about 1 mg/m² to about 9 mg/m² based on mirdametinib free base, about 1.5 mg/m² to about 8.5 mg/m² based on mirdametinib free base, about 2 mg/m² to about 8 mg/m² based on mirdametinib free base, about 2.5 mg/m² to about 7.5 mg/m² based on mirdametinib free base, about 3 mg/m² to about 7 mg/m² based on mirdametinib free base, about 3.5 mg/m² to about 6.5 mg/m² based on mirdametinib free base, about 4 mg/m² to about 6 mg/m² based on mirdametinib free base, or about 4.5 mg/m² to about 5.5 mg/m² based on mirdametinib free base. In some aspects, the mirdametinib, or a pharmaceutically acceptable salt thereof, is administered in a single dosage form comprising about 0.1 mg/m² based on mirdametinib free base, about 0.2 mg/m² based on mirdametinib free base, about 0.3 mg/m² based on mirdametinib free base, about 0.4 mg/m² based on mirdametinib free base, about 0.5 mg/m² based on mirdametinib free base, about 1 mg/m² based on mirdametinib free base, about 1.5 mg/m² based on mirdametinib free base, about 2 mg/m² based on mirdametinib free base, about 2.5 mg/m² based on mirdametinib free base, about 3 mg/m² based on mirdametinib free base, about 3.5 mg/m² based on mirdametinib free base, about 4 mg/m² based on mirdametinib free base, about 4.5 mg/m² based on mirdametinib free base, about 5 mg/m² based on mirdametinib free base, about 5.5 mg/m² based on mirdametinib free base, about 6 mg/m² based on mirdametinib free base, about 6.5 mg/m² based on mirdametinib free base, about 7 mg/m² based on mirdametinib free base, about 7.5 mg/m² based on mirdametinib free base, about 8 mg/m² based on mirdametinib free base, about 8.5 mg/m² based on mirdametinib free base, about 9 mg/m² based on mirdametinib free base, about 9.5 mg/m² based on mirdametinib free base, or about 10 mg/m² based on mirdametinib free base.

In some aspects, the mirdametinib, or a pharmaceutically acceptable salt thereof, is administered in a single dosage form comprising about 0.1 mg to about 10 mg based on mirdametinib free base, about 0.5 mg to about 9.5 mg based on mirdametinib free base, about 1 mg to about 9 mg based on mirdametinib free base, about 1.5 mg to about 8.5 mg based on mirdametinib free base, about 2 mg to about 8 mg based on mirdametinib free base, about 2.5 mg to about 7.5 mg based on mirdametinib free base, about 3 mg to about 7 mg based on mirdametinib free base, about 3.5 mg to about 6.5 mg based on mirdametinib free base, about 4 mg to about 6 mg based on mirdametinib free base, or about 4.5 mg to about 5.5 mg based on mirdametinib free base. In some aspects, the mirdametinib, or a pharmaceutically acceptable salt thereof, is administered in a single dosage form comprising about 0.1 mg based on mirdametinib free base, about 0.2 mg based on mirdametinib free base, about 0.3 mg based on mirdametinib free base, about 0.4 mg based on mirdametinib free base, about 0.5 mg based on mirdametinib free base, about 1 mg based on mirdametinib free base, about 1.5 mg based on mirdametinib free base, about 2 mg based on mirdametinib free base, about 2.5 mg based on mirdametinib free base, about 3 mg based on mirdametinib free base, about 3.5 mg based on mirdametinib free base, about 4 mg based on mirdametinib free base, about 4.5 mg based on mirdametinib free base, about 5 mg based on mirdametinib free base, about 5.5 mg based on mirdametinib free base, about 6 mg based on mirdametinib free base, about 6.5 mg based on mirdametinib free base, about 7 mg based on mirdametinib free base, about 7.5 mg based on mirdametinib free base, about 8 mg based on mirdametinib free base, about 8.5 mg based on mirdametinib free base, about 9 mg based on mirdametinib free base, about 9.5 mg based on mirdametinib free base, or about 10 mg based on mirdametinib free base.

In some aspects, the mirdametinib, or a pharmaceutically acceptable salt thereof, is administered one, two, three, or four times per day. In some aspects, the mirdametinib, or a pharmaceutically acceptable salt thereof, is administered once daily. In some aspects, the mirdametinib, or a pharmaceutically acceptable salt thereof, is administered twice daily.

In some aspects, the mirdametinib, or a pharmaceutically acceptable salt thereof, is administered twice daily in an amount of about 0.5 mg/m² to about 10 mg/m² based on mirdametinib free base, about 1 mg/m² to about 9.5 mg/m² based on mirdametinib free base, about 1.5 mg/m² to about 9 mg/m² based on mirdametinib free base, about 2 mg/m² to about 8.5 mg/m² based on mirdametinib free base, about 2.5 mg/m² to about 8 mg/m² based on mirdametinib free base, about 3 mg/m² to about 7.5 mg/m² based on mirdametinib free base, about 3.5 mg/m² to about 7 mg/m² based on mirdametinib free base, about 4 mg/m² to about 6.5 mg/m² based on mirdametinib free base, about 4.5 mg/m² to about 6 mg/m² based on mirdametinib free base, or about 5 mg/m² to about 6 mg/m² based on mirdametinib free base. In some aspects, the mirdametinib, or a pharmaceutically acceptable salt thereof, is administered twice daily in an amount of about 0.5 mg/m² based on mirdametinib free base, about 1 mg/m² based on mirdametinib free base, about 1.5 mg/m² based on mirdametinib free base, about 2 mg/m² based on mirdametinib free base, about 2.5 mg/m² based on mirdametinib free base, about 3 mg/m² based on mirdametinib free base, about 3.5 mg/m² based on mirdametinib free base, about 4 mg/m² based on mirdametinib free base, about 4.5 mg/m² based on mirdametinib free base, about 5 mg/m² based on mirdametinib free base, about 5.5 mg/m² based on mirdametinib free base, about 6 mg/m² based on mirdametinib free base, about 6.5 mg/m² based on mirdametinib free base, about 7 mg/m² based on mirdametinib free base, about 7.5 mg/m² based on mirdametinib free base, about 8 mg/m² based on mirdametinib free base, about 8.5 mg/m² based on mirdametinib free base, about 9 mg/m² based on mirdametinib free base, about 9.5 mg/m² based on mirdametinib free base, or about 10 mg/m² based on mirdametinib free base.

In some aspects, the mirdametinib, or a pharmaceutically acceptable salt thereof, is administered twice daily in an amount of about 0.5 mg to about 10 mg based on mirdametinib free base, about 1 mg to about 9.5 mg based on mirdametinib free base, about 1.5 mg to about 9 mg based on mirdametinib free base, about 2 mg to about 8.5 mg based on mirdametinib free base, about 2.5 mg to about 8 mg based on mirdametinib free base, about 3 mg to about 7.5 mg based on mirdametinib free base, about 3.5 mg to about 7 mg based on mirdametinib free base, about 4 mg to about 6.5 mg based on mirdametinib free base, about 4.5 mg to about 6 mg based on mirdametinib free base, or about 5 mg to about 6 mg based on mirdametinib free base. In some aspects, the mirdametinib, or a pharmaceutically acceptable salt thereof, is administered twice daily in an amount of about 0.5 mg based on mirdametinib free base, about 1 mg based on mirdametinib free base, about 1.5 mg based on mirdametinib free base, about 2 mg based on mirdametinib free base, about 2.5 mg based on mirdametinib free base, about 3 mg based on mirdametinib free base, about 3.5 mg based on mirdametinib free base, about 4 mg based on mirdametinib free base, about 4.5 mg based on mirdametinib free base, about 5 mg based on mirdametinib free base, about 5.5 mg based on mirdametinib free base, about 6 mg based on mirdametinib free base, about 6.5 mg based on mirdametinib free base, about 7 mg based on mirdametinib free base, about 7.5 mg based on mirdametinib free base, about 8 mg based on mirdametinib free base, about 8.5 mg based on mirdametinib free base, about 9 mg based on mirdametinib free base, about 9.5 mg based on mirdametinib free base, or about 10 mg based on mirdametinib free base.

In some aspects, the mirdametinib, or a pharmaceutically acceptable salt thereof, is administered in a total daily dose that does not exceed about 10 mg/m² based on mirdametinib free base, about 9.5 mg/m² based on mirdametinib free base, about 9 mg/m² based on mirdametinib free base, about 8.5 mg/m² based on mirdametinib free base, about 8 mg/m² based on mirdametinib free base, about 7.5 mg/m² based on mirdametinib free base, about 7 mg/m² based on mirdametinib free base, about 6.5 mg/m² based on mirdametinib free base, about 6 mg/m² based on mirdametinib free base, about 5.5 mg/m² based on mirdametinib free base, about 5 mg/m² based on mirdametinib free base, about 4.5 mg/m² based on mirdametinib free base, about 4 mg/m² based on mirdametinib free base, about 3.5 mg/m² based on mirdametinib free base, about 3 mg/m² based on mirdametinib free base, about 2.5 mg/m² based on mirdametinib free base, about 2 mg/m² based on mirdametinib free base, or about 1.5 mg/m² based on mirdametinib free base.

In some aspects, the mirdametinib, or a pharmaceutically acceptable salt thereof, is administered in a total daily dose that does not exceed about 10 mg based on mirdametinib free base, about 9.5 mg based on mirdametinib free base, about 9 mg based on mirdametinib free base, about 8.5 mg based on mirdametinib free base, about 8 mg based on mirdametinib free base, about 7.5 mg based on mirdametinib free base, about 7 mg based on mirdametinib free base, about 6.5 mg based on mirdametinib free base, about 6 mg based on mirdametinib free base, about 5.5 mg based on mirdametinib free base, about 5 mg based on mirdametinib free base, about 4.5 mg based on mirdametinib free base, about 4 mg based on mirdametinib free base, about 3.5 mg based on mirdametinib free base, about 3 mg based on mirdametinib free base, about 2.5 mg based on mirdametinib free base, about 2 mg based on mirdametinib free base, or about 1.5 mg based on mirdametinib free base.

In some aspects, the mirdametinib, or a pharmaceutically acceptable salt thereof, is administered as mirdametinib free base.

Methods for treating a patient having cutaneous neurofibromas (“cNF”), comprising administering to a patient in need thereof mirdametinib free base are provided herein. In some aspects, patient additionally has neurofibromatosis 1 (“NF1”).

In some aspects, a therapeutically effective amount of mirdametinib free base is administered.

In some aspects, the mirdametinib free base is administered in an amount of about 1 mg/m² to about 10 mg/m² per day, about 1.5 mg/m² to about 9.5 mg/m² per day, about 2 mg/m² to about 9 mg/m² per day, about 2.5 mg/m² to about 8.5 mg/m² per day, about 3 mg/m² to about 8 mg/m² per day, about 3.5 mg/m² to about 7.5 mg/m² per day, about 4 mg/m² to about 7 mg/m² per day, about 4.5 mg/m² to about 6.5 mg/m² per day, or about 5 mg/m² to about 6 mg/m² per day. In some aspects, the mirdametinib free base is administered in an amount of about 1 mg/m² per day, about 1.5 mg/m² per day, about 2 mg/m² per day, about 2.5 mg/m² per day, about 3 mg/m² per day, about 3.5 mg/m² per day, about 4 mg/m² per day, about 4.5 mg/m² per day, about 5 mg/m² per day, about 5.5 mg/m² per day, about 6 mg/m² per day, about 6.5 mg/m² per day, about 7 mg/m² per day, about 7.5 mg/m² per day, about 8 mg/m² per day, about 8.5 mg/m² per day, about 9 mg/m² per day, about 9.5 mg/m² per day, or about 10 mg/m² per day.

In some aspects, the mirdametinib free base is administered in an amount of about 1 mg to about 10 mg per day, about 1.5 mg to about 9.5 mg per day, about 2 mg to about 9 mg per day, about 2.5 mg to about 8.5 mg per day, about 3 mg to about 8 mg per day, about 3.5 mg to about 7.5 mg per day, about 4 mg to about 7 mg per day, about 4.5 mg to about 6.5 mg per day, or about 5 mg to about 6 mg per day. In some aspects, the mirdametinib free base is administered in an amount of about 1 mg per day, about 1.5 mg per day, about 2 mg per day, about 2.5 mg per day, about 3 mg per day, about 3.5 mg per day, about 4 mg per day, about 4.5 mg per day, about 5 mg per day, about 5.5 mg per day, about 6 mg per day, about 6.5 mg per day, about 7 mg per day, about 7.5 mg per day, about 8 mg per day, about 8.5 mg per day, about 9 mg per day, about 9.5 mg per day, or about 10 mg per day.

In some aspects, the mirdametinib free base is administered in a single dosage form comprising about 0.1 mg/m² to about 10 mg/m², about 0.5 mg/m² to about 9.5 mg/m², about 1 mg/m² to about 9 mg/m², about 1.5 mg/m² to about 8.5 mg/m², about 2 mg/m² to about 8 mg/m², about 2.5 mg/m² to about 7.5 mg/m², about 3 mg/m² to about 7 mg/m², about 3.5 mg/m² to about 6.5 mg/m², about 4 mg/m² to about 6 mg/m², or about 4.5 mg/m² to about 5.5 mg/m². In some aspects, the mirdametinib free base is administered in a single dosage form comprising about 0.1 mg/m², about 0.2 mg/m², about 0.3 mg/m², about 0.4 mg/m², about 0.5 mg/m², about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², or about 10 mg/m².

In some aspects, the mirdametinib free base is administered in a single dosage form comprising about 0.1 mg to about 10 mg, about 0.5 mg to about 9.5 mg, about 1 mg to about 9 mg, about 1.5 mg to about 8.5 mg, about 2 mg to about 8 mg, about 2.5 mg to about 7.5 mg, about 3 mg to about 7 mg, about 3.5 mg to about 6.5 mg, about 4 mg to about 6 mg, about 4.5 mg to about 5.5 mg, or about 5 mg to about 6 mg. In some aspects, the mirdametinib free base is administered in a single dosage form comprising about 0.1 mg, about 0.2 mg, about 0.3 mg, about 0.4 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg.

In some aspects, the mirdametinib free base is administered one, two, three, or four times per day. In some aspects, the mirdametinib free base is administered once daily. In some aspects, the mirdametinib free base is administered twice daily.

In some aspects, the mirdametinib free base is administered twice daily in an amount of about 0.5 mg/m² to about 10 mg/m², about 1 mg/m² to about 9.5 mg/m², about 1.5 mg/m² to about 9 mg/m², about 2 mg/m² to about 8.5 mg/m², about 2.5 mg/m² to about 8 mg/m², about 3 mg/m² to about 7.5 mg/m², about 3.5 mg/m² to about 7 mg/m², about 4 mg/m² to about 6.5 mg/m², about 4.5 mg/m² to about 6 mg/m², or about 5 mg/m² to about 6 mg/m². In some aspects, the mirdametinib free base is administered twice daily in an amount of about 0.5 mg/m², about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², or about 10 mg/m².

In some aspects, the mirdametinib free base is administered twice daily in an amount of about 0.5 mg to about 10 mg, about 1 mg to about 9.5 mg, about 1.5 mg to about 9 mg, about 2 mg to about 8.5 mg, about 2.5 mg to about 8 mg, about 3 mg to about 7.5 mg, about 3.5 mg to about 7 mg, about 4 mg to about 6.5 mg, about 4.5 mg to about 6 mg, or about 5 mg to about 6 mg. In some aspects, the mirdametinib free base is administered twice daily in an amount of about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg.

In some aspects, the mirdametinib free base is administered in a total daily dose that does not exceed about 10 mg/m², about 9.5 mg/m², about 9 mg/m², about 8.5 mg/m², about 8 mg/m², about 7.5 mg/m², about 7 mg/m², about 6.5 mg/m², about 6 mg/m², about 5.5 mg/m², about 5 mg/m², about 4.5 mg/m², about 4 mg/m², about 3.5 mg/m², about 3 mg/m², about 2.5 mg/m², about 2 mg/m², or about 1.5 mg/m².

In some aspects, the mirdametinib free base is administered in a total daily dose that does not exceed about 10.5 mg, 10 mg, about 9.5 mg, about 9 mg, about 8.5 mg, about 8 mg, about 7.5 mg, about 7 mg, about 6.5 mg, about 6 mg, about 5.5 mg, about 5 mg, about 4.5 mg, about 4 mg, about 3.5 mg, about 3 mg, about 2.5 mg, about 2 mg, or about 1.5 mg.

In yet another embodiment, the mirdametinib, or a pharmaceutically acceptable salt thereof, is orally administered in an amount of about 2 mg/m² per day based on mirdametinib free base.

In yet another embodiment, the mirdametinib, or a pharmaceutically acceptable salt thereof, is orally administered in an amount of about 2 mg per day based on mirdametinib free base.

In yet another embodiment, the mirdametinib, or a pharmaceutically acceptable salt thereof, is orally administered in an amount of about 4 mg per day based on mirdametinib free base.

In yet another embodiment, the mirdametinib, or a pharmaceutically acceptable salt thereof, is orally administered in an amount of about 6 mg per day based on mirdametinib free base.

In yet another embodiment, the mirdametinib, or a pharmaceutically acceptable salt thereof, is orally administered in an amount of about 8 mg per day based on mirdametinib free base.

In yet another embodiment, the mirdametinib, or a pharmaceutically acceptable salt thereof, is orally administered twice daily in an amount of about 1 mg based on mirdametinib free base.

In yet another embodiment, the mirdametinib, or a pharmaceutically acceptable salt thereof, is orally administered twice daily in an amount of about 2 mg based on mirdametinib free base.

In yet another embodiment, the mirdametinib, or a pharmaceutically acceptable salt thereof, is orally administered twice daily in an amount of about 3 mg based on mirdametinib free base.

In yet another embodiment, the mirdametinib, or a pharmaceutically acceptable salt thereof, is orally administered twice daily in an amount of about 4 mg based on mirdametinib free base.

In one embodiment of any of the methods described herein,
(a) for a patient having a body surface area no more than 0.69 m², the patient is initially orally administered 1 mg mirdametinib twice daily,
(b) for a patient having a body surface area of 0.7 to 1.04 m², the patient is initially orally administered 2 mg mirdametinib twice daily,
(c) for a patient having a body surface area of 1.05 to 1.49 m², the patient is initially orally administered 3 mg mirdametinib twice daily, and
(d) for a patient having a body surface area of at least 1.5 m², the patient is initially orally administered 4 mg mirdametinib twice daily.

In one embodiment, the maximum daily dose is 4 mg mirdametinib twice daily.

In one embodiment, over each four week period, the mirdametinib is administered for the first three weeks and discontinued for the last one week.

In another embodiment, the mirdametinib is administered daily without interruption.

In one embodiment of any of the methods described herein, the dose administered is reduced due to an adverse event, wherein the dose is reduced as follows:
(a) if the dose at the time of the event is 1 mg mirdametinib twice daily, then the reduced daily dose is 1 mg orally administered in the morning only;
(b) if the dose at the time of the event is 2 mg mirdametinib twice daily, then the reduced daily dose is 2 mg orally administered in the morning and 1 mg administered in the afternoon or evening;
(c) if the dose at the time of the event is 3 mg mirdametinib twice daily, then the reduced daily dose is 2 mg orally administered twice daily; and
(d) if the dose at the time of the event is 4 mg mirdametinib twice daily, then the reduced daily dose is 3 mg orally administered twice daily. In one embodiment, the adverse event resulting in the dose reduction is acneiform.

In one embodiment of any of the methods described herein, the method further comprises prior to treatment (i) determining whether to select mirdametinib as a treatment for the patient, and (ii) selecting mirdametinib as a treatment for the patient at least partially based on its objective response rate, where the objective response rate is defined as at least a 20% decrease in tumor size using centrally read MRI volumetric analysis. In one embodiment, in step (i), mirdametinib is selected based on a response rate of at least 70%. In another embodiment, in step (i), mirdametinib is selected based on a response rate of at least 75%. In yet another embodiment, in step (i), mirdametinib is selected based on a response rate of at least 80%. In yet another embodiment, in step (i), mirdametinib is selected based on a response rate of at least 85%. In yet another embodiment, in step (i), mirdametinib is selected based on a response rate of at least 90%. In yet another embodiment, in step (i), mirdametinib is selected based on a response rate of at least 95%.

In some aspects, the mirdametinib, or a pharmaceutically acceptable salt thereof, exhibits high blood-brain-barrier penetration.

In one embodiment, the patient has the clinical diagnosis of NF1 using the NIH Consensus Conference and one or more of the following:
(a) six or more café-au-lait macules with a diameter >5 mm in prepubertal and >15 mm in post-pubertal individuals;
(b) freckling in axilla or inguinal regions;
(c) optic glioma;
(d) two or more Lisch nodules;
(e) a distinctive bony lesion (dysplasia of the sphenoid bone or dysplasia of thinning of long bone cortex); and
(f) a first degree relative with NF1.

In another embodiment, the patient has a constitutional NF1 mutation documented in a Clinical Laboratory Improvement Amendments/College of American Pathologists certified lab.

In one embodiment of any of the methods described herein, the patient is a human. In one embodiment, the human is an adult. In another embodiment, the patient is 2 to 15 years of age. In some aspects, the human has an age of ≥2 and ≤18 years.

In some aspects, the human has had no prior exposure to MEK inhibitors. In some aspects, the human has not responded to prior treatment to one or more MEK inhibitors.

In some aspects, the mirdametinib, or a pharmaceutically acceptable salt thereof, is administered orally. In some aspects, the mirdametinib, or a pharmaceutically acceptable salt thereof, is administered orally as a solid dosage form. In some aspects, the solid dosage form is a tablet or capsule. In some aspects, the solid dosage form is a capsule. In some aspects, the mirdametinib, or a pharmaceutically acceptable salt thereof, is dispersible in a potable liquid or orodispersible in a patient's saliva.

In some aspects, the mirdametinib, or a pharmaceutically acceptable salt thereof, is administered as a monotherapy to treat the cutaneous neurofibromas (“cNF”).

In some aspects, the mirdametinib, or a pharmaceutically acceptable salt thereof, is administered in combination with another active ingredient and/or surgery to treat the cutaneous neurofibromas (“cNF”).

### EXAMPLE

### Example 1: Open-label, Multicenter Dose Finding Study to Evaluate the Safety and Tolerability and Anti-Tumor Activity of Mirdametinib Monotherapy in Adult Patients with Neurofibromatosis 1 (NF1) and Cutaneous Neurofibromas (cNF)

A Phase 1/2a open-label, multicenter dose finding study to evaluate the safety and tolerability and anti-tumor activity of mirdametinib monotherapy in adult patients with neurofibromatosis 1 (NF1) and cutaneous neurofibromas (cNF).

Phase 1 Study Objectives: The primary study objective is to assess the safety and tolerability of mirdametinib monotherapy in adult patients with NF1 and cNF and determine the RP2D (recommended phase 2 dose). The secondary study objective is to evaluate the anti-tumor activity of mirdametinib monotherapy in adult patients with NF1 and cNF. The exploratory study objectives are to assess the on-treatment effect of mirdametinib on target inhibition (PERK, markers of apoptosis, mitogenetic markers and measures of the extra-cellular matrix proteins) in cutaneous neurofibroma(s) in adult patients with cutaneous neurofibromas (cNF) and NF1.

Phase 2A (POC) Study Objectives: The primary study objective is to assess the safety of mirdametinib monotherapy in adult patients with NF1 and cNF. The secondary study objective is to assess the efficacy of mirdametinib monotherapy in adult patients with NF1 and cNF. The exploratory study objectives are (1) to assess the quality-of-life impact of mirdametinib monotherapy in adult patients with NF1 and cNF; and (2) assess the on-treatment effect of mirdametinib on target inhibition (PERK, markers of apoptosis, mitogenetic markers and measures of the extra-cellular matrix proteins) in cutaneous neurofibroma(s) in adult patients with cutaneous neurofibromas (cNF) and NF1.

### Study Design:

The study will be conducted in two phases: 1) Phase 1, will test the safety and tolerability of multiple dose cohorts of mirdametinib to identify up to two doses for Phase 2a; 2) Phase 2a of the study will test the efficacy and safety of up to two different doses in participants with NF1 and cNF.

In both Phases of the study, participation in the study will comprise three periods: screening, treatment and post-study safety follow-up.

### 1. Screening Period (Phases 1 and 2a)

Participants will be screened for up to 28 days prior to first dose of study treatment. Participant's disease diagnosis will be confirmed using the clinical and pathology reports available for the participant at the time of screening initiation, written informed consent, clinical and laboratory investigations to establish the eligibility of the participant for the trial will be completed.

Cardiac, ophthalmologic and laboratory examination (including serum/urine pregnancy test), will be completed.

The two target areas will be chosen (7 cm×7 cm with a minimum of 6 cutaneous neurofibromas of more than ≥0.5 cm); 2D photograph of the target areas will be taken to ensure tracking of the same lesions.

This is a routine care visit during which a usual clinical examination is performed. Eligibility will be determined based on the screening visit.

The following patient-reported outcome measures will be completed: Skindex 26; Burden of Neurofibromatosis assessment tool; Visual Analogic Scale for pruritus;
For participants not enrolled, a brief reason will be entered.
Eligible participants will take their first dose of study treatment following all pre-dose assessments at Cycle 1 Day 1. All participants will remain in the Treatment Phase of the study until disease progression, they discontinue study treatment for any other reason, the study is stopped by the Sponsor for any reason, or the participant has completed the study through Cycle 12 for Phase 1 or Cycle 12 for Phase 2a.

### 2. Treatment Period

Phase 1 Study: Participants would have completed screening prior to the first dose of study treatment (Mirdametinib). There will be 4 dose groups each with a sample size of 6 participants.
1. Study treatment will be administered orally at a dose of 1 mg BID, 2 mg BID, 3 mg BID or 4 mg BID for the 4 Dose Groups, respectively.
2. Dosing will be on a 28-day Cycle (4-week course) with a continuous dosing schedule. The Treatment Phase will last for up to 12 Cycles followed by a 30-day Safety Follow-Up period (the Safety Follow-Up visit is only required for participants who are not continuing in the Phase 2a).
3. Study visits and participants evaluation will be as in the schedule of activities.

Phase 2a Study: Participants previously enrolled in the selected RP2D groups of the Phase 1 will be enrolled in the Phase 2 Study.
1. Participants newly enrolling into the Phase 2 will be screened for up to 28 days prior to the first dose of study treatment (Mirdametinib). Participants meeting enrollment criteria will be randomly assigned to either of the two active treatment groups or to placebo.
2. Each group will have 12 participants for a total of up to 36 participants in the study. Study treatment will be administered orally at a dose to be determined following the Phase 1 Study. Dosing will be on a 28-day Cycle (4-week course) with a continuous dosing schedule. The Treatment Phase will last for up to 12 Cycles followed by a 30-day Safety Follow-Up period.
3. Assessment of endpoints and PRO will be performed, every 3 months (Pre-treatment baseline, M3, M6, M9, M12).
4. Subjects will complete a daily diary to record adverse events and treatment adherence.

### Safety Follow-up Period (Phase 1 and Phase 2a):

The post-study follow-up (FU) visit will be performed at 30 days (+3 days) after the last dose of study drug, except for subjects who died, withdrew consent and objected to further data collection, received new treatment with a MEK-inhibitor or were lost to follow up.

The FU assessments can be done via a clinic visit (recommended) or via a telephone call (if subject cannot attend a clinic visit).

### Phase 1 (Dose Finding)

In this phase, four different dose options of mirdametinib will be tested in a maximum of 24 participants to identify the two doses to move into Phase 2. The two doses will be selected based on tolerability and efficacy signals. Each study arm will be enrolled and treated concurrently except for Dose Group 4. Dose Group 4 (4 mg BID continuous group) will not be initiated until the 3 mg BID dose has been deemed tolerable.

### Cohort Management During Dose Finding

The dose groups will proceed in parallel. The actual number of dose groups will be four; each group will have 6 participants for a total of 24 participants in this phase of the study.

TABLE 1
Doses per dose group in Stage 1

| Dose Group | Dose |
|---|---|
| 1 | 1 mg BID |
| 2 | 2 mg BID |
| 3 | 3 mg BID |
| 4 | 4 mg BID |

Each dose group will start with a continuous dosing approach. If 2 participants (33%) in a dose group develop a DLT (defined below) then that dose group will dose reduce to an intermittent (3 weeks on 1 week off) approach. If the DLTs do not resolve or if a further 33% (N=2) of participants experience DLTs on the intermittent dosing approach, then that dose group will be terminated.

The first management decision will be made after at least 25% of participants (N=6) complete C1 or experience a DLT.

### Definition of Dose-Limiting Toxicities (DLT)

DLT is defined as one or more of the following events occurring during the 28-day DLT period in Cycle 1, with the exclusion of toxicities clearly related to disease progression or intercurrent illness. The severity of DLTs will be graded according to the CTCAE v5.0.

### Hematologic:

Grade ≥4 anemia.
Hematologic toxicity as defined by:
   Grade 4 neutropenia lasting >5 days.
   Grade 3 febrile neutropenia (defined as absolute neutrophil count [ANC] <1000/mm³ with a single body temperature reading of >38.3° C. (>101° F.) or a sustained temperature of ≥38° C. (≥100.4° F.) for >1 hour.
   Grade 4 thrombocytopenia or grade 3 thrombocytopenia with clinically significant bleeding.

Non-hematologic:
Grade ≥3 ocular toxicities leading to reduced visual acuity limiting activities of daily living (ADL), including but not limited to glaucoma, retinal detachment, or retinal vascular disorder.
Symptomatic decrease in left ventricular ejection fraction (LVEF) or any other sign or symptom of congestive heart failure (CHF).
Events meeting the criteria for Hy's law as follows (all 3 features):
   Aspartate aminotransferase (AST) and/or alanine aminotransferase (ALT) >3×upper limit of normal (ULN)
   Concurrent elevation of total bilirubin >2×ULN without initial evidence of cholestasis (elevated serum alkaline phosphatase)
   No alternative etiology can be identified
Grade ≥3 significant neurologic toxicity (e.g., seizure, hallucination, confusion or delirium)
Grade ≥3 generalized muscle weakness.
Grade ≥3 CK increase, if associated with the significant increase in the skeletal muscle isoenzyme (CK-MM) confirmed by electrophoretic fractionation and after ruling out the factors unrelated to the study drug (e.g., physical exercise, trauma, inflammatory comorbidity).
Grade ≥3 skin rash that did not begin to resolve within 7 days of initiating optimal medical and supportive care.
Grade ≥3 non-hematologic toxicity not listed above (except alopecia) that resolve to Grade ≤1 within 3 days of initiating optimal medical and supportive therapy.

In addition, any clinically important or persistent toxicities that are not included above may also be considered a DLT following review and discussion between the CMC and the sponsor.

The following toxicities may not be considered a DLT at the investigator's discretion after consultation with the Medical Monitor
Isolated Grade 3 or Grade 4 laboratory abnormalities not listed above (e.g., lactose dehydrogenase elevation) that have no clinical correlate and resolve to Grade ≤1 within 3 days of initiating optimal medical and supportive therapy.
Grade ≥3 nausea, vomiting, diarrhea that resolves in ≤3 days, in the absence of optimal medical therapy.
Grade ≥3 fatigue that resolves in ≤5 days.
Grade ≥3 asymptomatic elevation in lipase or amylase without pancreatitis that lasts ≤3 days.

### Phase 2a (Safety and Efficacy POC)

The objective of this phase will be to generate POC data on the safety and anti-tumor efficacy of mirdametinib in treating cNF. In addition, PK/PD will be evaluated.

This Phase will start after 2 doses are selected from Phase 1 for further testing. Participants completing phase 1 on either of the two recommended Phase 2 doses will continue in Phase 2 and the cohorts will be expanded to 12 participants each.

### Tracking cNF Lesions

To ensure the same lesions are measured over time during the study, ‘Target Areas’ and ‘Target cNF’ lesions will be identified and marked at pre-treatment baseline.

Two ‘Target Areas’ (7 cm×7 cm with a minimum of 6 cutaneous neurofibromas ‘Target cNF’ lesions of ≥0.5 cm in their longest diameter) will be defined. 2D photography will be used to document the ‘Target cNF’ lesions in relation to cutaneous landmarks in combination with a permanent map of the ‘Target Area’ created by marking the location of each cNF on a plastic sheet template placed over the participant's relevant body region.

### Measuring cNF Lesions

The study will use handheld 3D camera and digital calipers as measuring tools for the cNF lesions.

In screening, 3D photography or digital calipers (as locally preferred) will be used to evaluate the size of the cNF lesions for eligibility.

In Phase 1, both 3D photography and digital calipers will be used to assess tumor response.

In Phase 2a, the primary efficacy assessment will be based on the 3D camera and the digital calipers will be used as a secondary endpoint.

To minimize variability in measurements, the same technician/staff will be used to acquire 3D images or caliper measurements throughout the trial. A blinded central reviewer will be used for 3D image analysis. All photographs at each time point should be taken from the same distance, using similar lighting and camera settings.

### Study Population

Eligible patients will be adult patients (male and/or non-pregnant females), ≥18 years of age, confirmed with NF1, and who have a minimum of 12 measurable cutaneous neurofibromas (defined as non-pedunculated, surrounded by normal skin and not adjacent to another cNF lesion), each measuring ≥0.5 cm in the longest diameter and ≥0.5 cm in height, across 2 ‘Target Areas’ (defined as any 2 of the following body regions: head and neck; upper extremities; anterior chest wall; posterior chest wall, anterior abdominal wall; lower back; pelvic region/gluteal region; lower extremities).

### Eligibility Inclusion

### Inclusion Criteria:

Participants must meet ALL the following criteria to be eligible for the study.
1. Participants must have EITHER the clinical diagnosis of NF1 using the National Institute of Health (NIH) Consensus Conference criteria of at least 1 other diagnostic criterion (Inclusion 2.1-2.6, see below) in addition to the presence of plexiform neurofibroma (PN), OR have a constitutional NF1 mutation documented in a Clinical Laboratory Improvement Amendments/College of American Pathologists certified lab; additional criteria are as follows:
2.1. Six or more café-au-lait macules with a diameter >5 mm in prepubertal and >15 mm in post-pubertal individuals, respectively
2.2. Freckling in axilla or inguinal regions;
2.3. Optic glioma;
2.4. Two or more Lisch nodules;
2.5. A distinctive bony lesion (dysplasia of the sphenoid bone or dysplasia of thinning of long bone cortex);
2.6. A first degree relative with NF1;
3. Participants must have a Karnofsky performance level of ≥80%.
4. Participant has adequate organ and bone marrow function as defined by the following Screening laboratory values:
   4.1 Absolute neutrophil count ≥1500 cells/μL;
   4.2. Platelets ≥100×10³/μL;
   4.3. Hemoglobin ≥9.5 g/dL;
   4.4. Serum albumin ≥2.8 g/dL;
   4.5. Calculated creatinine clearance at Screening ≥60 mL/min (by Cockcroft-Gault formula) OR a normal serum creatinine.
5. Participant has the ability to swallow capsules whole if the capsule dosage form is being utilized. This criterion does not apply if participant is utilizing the dispersible tablet (Pediatric Dosage formulation) dosage form of study treatment;
6. Participant is willing and able to comply with all aspects of the protocol;

### Contraception

7. Male or Female Contraceptive use by men or women should be consistent with local regulations regarding the methods of contraception for those participating in clinical studies.
   a. Male participants:
   Male participants are eligible to participate if they agree to the following during the treatment period and for at least 90 days after the last dose of study treatment:
      Refrain from donating sperm.
   PLUS either:
      Be abstinent from heterosexual intercourse as their preferred and usual lifestyle (abstinent on a long term and persistent basis) and agree to remain abstinent
   OR
      Must agree to use a male condom when having sexual intercourse with a woman of child bearing potential (WOCBP).
   b. Female participants:
   Female participants are eligible to participate if they are not pregnant or breastfeeding, and at least one of the following conditions applies:
      Is not a woman of childbearing potential
         OR
      Is a WOCBP and using a contraceptive method that is highly effective (with a failure rate of <1% per year), preferably with low user dependency during the treatment period and for at least 30 days after the last dose of study treatment and agrees not to donate eggs (ova, oocytes) for the purpose of reproduction during the study and for a period of 90 days after last dose of study treatment. The Investigator should evaluate the effectiveness of the contraceptive method in relationship to the first dose of study treatment.
         A WOCBP must have a negative serum pregnancy test result at Screening and a negative urine pregnancy test result at the Baseline visit prior to the first dose of study treatment.
      The Investigator is responsible for review of medical history, menstrual history, and recent sexual activity to decrease the risk for inclusion of a woman with an early undetected pregnancy;

### Informed Consent/Assent

The Investigator, or a person designated by the Investigator, will obtain written informed consent from each study participant or the participant's legally acceptable representative, parent(s), or legal guardian and the participant's assent, when applicable, before any study-specific activity is performed. The Investigator will retain the original copy of each participant's signed consent/assent document

Exclusion criteria:

Participants who meet ANY of the following criteria will not be eligible for the study.

### Medical Conditions

Participant has a Screening alanine transaminase (ALT) value of >2.0×upper limit of normal (ULN);

Participant has a Screening total bilirubin value of >1.5×ULN (isolated bilirubin >1.5×ULN is acceptable if bilirubin is fractionated and direct bilirubin <35%);

Participant has a history of malignancy associated hypercalcemia;

Participant has an active parathyroid disorder, hyperphosphatemia at Screening (serum phosphorus >1×ULN), or serum calcium (mg/dL) x serum phosphorus (mg/dL) product >70 at Screening;

Any clinically significant active or known history of liver disease, or known hepatic or biliary abnormalities (with the exception of Gilbert's syndrome or asymptomatic gallstones);
a. Hepatitis serology and viral load will be tested at Screening. Patients who are hepatitis B surface antigen (HBsAg) positive or hepatitis C virus (HCV) antibody positive at Screening must not be enrolled until further definite testing with hepatitis B virus (HBV) deoxyribonucleic acid (DNA) titers is <500 IU/mL or HCV ribonucleic acid (RNA) polymerase chain reaction test is negative;

Lymphoma, leukemia, or any malignancy (including malignant glioma or MPNST) within the past 5 years except for basal cell or squamous epithelial carcinomas of the skin that have been resected with no evidence of metastatic disease for 3 years;

Breast cancer within the past 10 years;

Participants with evidence of an active optic glioma or other low-grade glioma, requiring treatment with chemotherapy or radiation therapy. Participants not requiring treatment are eligible. Ophthalmological findings secondary to long-standing optic pathway glioma (such as visual loss, optic nerve pallor or strabismus) or long-standing orbito-temporal PN (such as visual loss, strabismus) will NOT be considered a significant abnormality for the purposes of the study;

Participant has abnormal QT interval corrected by Fridericia's formula (>450 msec for male participants, >470 msec for female participants, or >480 msec for participants with bundle branch block) (triplicate ECG readings taken approximately 2 to 3 minutes apart and averaged) at Screening;

Participant has experienced any of the following within 6 months (24 weeks) of signing informed consent/assent: clinically significant cardiac disease, myocardial infarction, severe/unstable angina, coronary/peripheral artery bypass graft, cerebrovascular accident, transient ischemic attack, or symptomatic pulmonary embolism;

Participant has recorded a LVEF <55% at Screening or within 3 years of signing informed consent/assent, OR has a history of congestive heart failure;

Participant has a history of, or evidence of, retinal pathology on

ophthalmologic examination that is considered a risk factor for central serous retinopathy, retinal vein occlusion (RVO), or neovascular macular degeneration. Participants will be excluded from study participation if they have any of the following risk factors for RVO at Screening:
a. Intraocular pressure >21 mmHg;
b. Serum cholesterol >300 mg/dL;
c. Serum triglycerides >300 mg/dL;
d. Hyperglycemia (fasting blood glucose >125 mg/dL or random blood glucose >200 mg/dL);
e. Age specific hypertension
   i. Participants ≥13 years of age with a blood pressure ≥140/90 mm Hg
   ii. Participants ≤12 years of age with a blood pressure ≥95^{th} percentile for age +12 mmHg;

Participant has a history of glaucoma;

Participant has a history of a positive human immunodeficiency virus (HIV) antibody test;

Participant has a known malabsorption syndrome or preexisting gastrointestinal conditions that may impair absorption of mirdametinib (e.g., gastric bypass, lap band, or other gastric procedures). Delivery of mirdametinib via nasogastric tube or gastrostomy tube is not allowed;

### Prior/Concomitant Therapy

Participant previously received or is currently receiving therapy with mirdametinib;

Participant is receiving systemic or ocular glucocorticoid therapy (with the exception of participants with endocrine deficiencies who are allowed to receive physiologic or stress doses of steroids, if necessary) within 14 days prior to first dose of study treatment;

Participant has received radiation therapy within the 6 months prior to signing of informed consent/assent. Participants who have received radiation to the orbit at any time are excluded;

### Prior/Concurrent Clinical Study Experience

Current enrollment or past participation in any other clinical study (excluding observational studies) within 28 days of signing of informed consent/assent;

### Other Exclusions

Sensitivity to the study treatment, or components thereof, or drug or other allergy that, in the opinion of the Investigator or Medical Monitor, contraindicates participation in the study;

Participant with active bacterial, fungal, or viral infection including but not limited to the use of antibiotics, antifungals, or antiviral agents at the time of Screening;

Underlying medical conditions, laboratory abnormality, or alcohol or drug abuse or dependence that, in the Investigator's opinion, will be unfavorable for the administration of study treatment or affect the explanation of drug toxicity or adverse events; or insufficient compliance during the study according to Investigator's judgement; or

Participant has experienced other severe acute or chronic medical or psychiatric conditions, including recent (within 1 year of signing informed consent/assent) or active suicidal ideation or behavior, or a laboratory abnormality that may increase the risk associated with study participation or study treatment administration or may interfere with the interpretation of study results and, in the judgment of the Investigator, would make the participant inappropriate for entry into this study.

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations following, in general, the principles and including such departures from the present disclosure that come within known or customary practice within the art to which the invention pertains and can be applied to the essential features hereinbefore set forth, and follows in the scope of the claimed.

1. A method for treating cutaneous neurofibromas (cNF) comprising administering to a human patient in need thereof, mirdametinib or a pharmaceutically acceptable salt thereof.

2. The method of claim 1, wherein the patient additionally has neurofibromatosis 1 (NF1).

3. The method of claim 1, wherein the patient has the clinical diagnosis of NF1 using the NIH Consensus Conference and one or more of the following:
(a) six or more café-au-lait macules with a diameter >5 mm in prepubertal and >15 mm in post-pubertal individuals;
(b) freckling in axilla or inguinal regions;
(c) optic glioma;
(d) two or more Lisch nodules;
(e) a distinctive bony lesion (dysplasia of the sphenoid bone or dysplasia of thinning of long bone cortex); and
(f) a first degree relative with NF1.

4. The method of claim 1, wherein the patient has a constitutional NF1 mutation documented in a Clinical Laboratory Improvement Amendments/College of American Pathologists certified lab.

5. The method of claim 1, wherein a therapeutically effective amount of mirdametinib, or a pharmaceutically acceptable salt thereof, is administered.

6. The method of claim 1, wherein the mirdametinib, or a pharmaceutically acceptable salt thereof, is orally administered in an amount of about 1 mg/m² to about 10 mg/m² per day based on mirdametinib free base.

7. The method of claim 1, wherein the mirdametinib, or a pharmaceutically acceptable salt thereof, is orally administered in an amount of about 1 mg to about 10 mg per day based on mirdametinib free base.

8. The method of claim 1, wherein the mirdametinib, or a pharmaceutically acceptable salt thereof, is administered once daily.

9. The method of claim 1, wherein the mirdametinib, or a pharmaceutically acceptable salt thereof, is administered twice daily.

10. The method of claim 1, wherein the mirdametinib, or a pharmaceutically acceptable salt thereof, is orally administered in an amount of about 2 mg/m² per day based on mirdametinib free base.

11. The method of claim 1, wherein the mirdametinib, or a pharmaceutically acceptable salt thereof, is orally administered in an amount of about 2 mg per day based on mirdametinib free base.

12. The method of claim 1, wherein the mirdametinib, or a pharmaceutically acceptable salt thereof, is orally administered in an amount of about 4 mg per day based on mirdametinib free base.

13. The method of claim 1, wherein the mirdametinib, or a pharmaceutically acceptable salt thereof, is orally administered in an amount of about 6 mg per day based on mirdametinib free base.

14. The method of claim 1, wherein the mirdametinib, or a pharmaceutically acceptable salt thereof, is orally administered in an amount of about 8 mg per day based on mirdametinib free base.

15. The method of claim 1, wherein the mirdametinib, or a pharmaceutically acceptable salt thereof, is orally administered twice daily in an amount of about 1 mg based on mirdametinib free base.

16. The method of claim 1, wherein the mirdametinib, or a pharmaceutically acceptable salt thereof, is orally administered twice daily in an amount of about 2 mg based on mirdametinib free base.

17. The method of claim 1, wherein the mirdametinib, or a pharmaceutically acceptable salt thereof, is orally administered twice daily in an amount of about 3 mg based on mirdametinib free base.

18. The method of claim 1, wherein the mirdametinib, or a pharmaceutically acceptable salt thereof, is orally administered twice daily in an amount of about 4 mg based on mirdametinib free base.

19. The method of claim 1, wherein
(a) for a patient having a body surface area no more than 0.69 m², the patient is initially orally administered 1 mg mirdametinib twice daily,
(b) for a patient having a body surface area of 0.7 to 1.04 m², the patient is initially orally administered 2 mg mirdametinib twice daily,
(c) for a patient having a body surface area of 1.05 to 1.49 m², the patient is initially orally administered 3 mg mirdametinib twice daily, and
(d) for a patient having a body surface area of at least 1.5 m², the patient is initially orally administered 4 mg mirdametinib twice daily.

20. The method of claim 1, wherein the maximum daily dose is 4 mg mirdametinib twice daily.

21. The method of claim 1, wherein over each four week period, the mirdametinib is administered for the first three weeks and discontinued for the last one week.

22. The method of claim 1, wherein the dose administered is reduced due to an adverse event, wherein the dose is reduced as follows:
(a) if the dose at the time of the event is 1 mg mirdametinib twice daily, then the reduced daily dose is 1 mg orally administered in the morning only;
(b) if the dose at the time of the event is 2 mg mirdametinib twice daily, then the reduced daily dose is 2 mg orally administered in the morning and 1 mg administered in the afternoon or evening;
(c) if the dose at the time of the event is 3 mg mirdametinib twice daily, then the reduced daily dose is 2 mg orally administered twice daily; and
(d) if the dose at the time of the event is 4 mg mirdametinib twice daily, then the reduced daily dose is 3 mg orally administered twice daily.

23. The method of claim 22, wherein the adverse event resulting in the dose reduction is acneiform.

24. The method of claim 1, wherein the method further comprises prior to treatment (i) determining whether to select mirdametinib as a treatment for the patient, and (ii) selecting mirdametinib as a treatment for the patient at least partially based on its objective response rate, where the objective response rate is defined as at least a 20% decrease in tumor size using centrally read MRI volumetric analysis.

25. The method of claim 25, wherein in step (i), mirdametinib is selected based on a response rate of at least 70%.

26. The method of claim 1, wherein the patient is 2 to 15 years of age.

27. The method of claim 1, wherein the human is an adult.

28. The method of claim 1, wherein the human patient has no prior exposure to MEK inhibitors.

29. The method of claim 1, wherein the mirdametinib, or a pharmaceutically acceptable salt thereof, is administered as a monotherapy.

30. The method of claim 1, wherein the mirdametinib, or a pharmaceutically acceptable salt thereof, is administered in combination with another active ingredient and/or surgery.
